(19) United States
(12) Patent Application Publication
(10) Pub. No.: US 2009/0209845 A1
(43) Pub. Date: Aug. 20, 2009

(54) METHOD TO OPTIMIZE INTERACTIVE PRODUCTS BASED ON THEIR FUNCTIONAL NEURAL IMPACT

(76) Inventors: Patricia L. CHRISTEN, Piedmont, CA (US); Steve W. Cole, San Carlos, CA (US)

Correspondence Address: SHAY GLENN LLP, 2755 CAMPUS DRIVE, SUITE 210, SAN MATEO, CA, 94403, US

(21) Appl. No.: 12/390,297

(22) Filed: Feb. 20, 2009

Related U.S. Application Data
(60) Provisional application No. 61/030,151, filed on Feb. 20, 2008.

(51) Int. Cl. A61B 5/0482; A63F 13/10; A61B 5/055; A63F 13/00

(52) U.S. Cl. 600/411; 463/36; 600/545

(57) ABSTRACT

Described herein are methods of optimizing an interactive media (e.g., a videogame) based on a brain activity criterion, as well as interactive media optimized or incorporating these methods. Brain activity may be measured using imaging techniques, such as an fMRI image of neural activity taken while a subject is using the technology. The optimization may be used to help the interactive media evoke desired behaviors or attitudes in subjects who have used the interactive media. For example, the interactive media described herein may be optimized to help facilitate behaviors or attitudes that are conducive to medical therapies, including compliance with medical treatments.

### CROSS REFERENCE TO RELATED APPLICATIONS

This patent claims priority to U.S. Provisional Patent application Ser. No. 61/030151, filed Feb. 20, 2008, titled “A METHOD TO OPTIMIZE INTERACTIVE PRODUCTS BASED ON THEIR FUNCTIONAL NEURAL IMPACT”.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Interactive digital media, such as internet sites (e.g., webpages), videogames, and toys, may be used to amuse, to educate, and may also help entrain desired behaviors or attitudes. Interactive media may help a user perform better in a therapy, task or situation many days, or even months, after the user has engaged the interactive media. For example, “Re-mission” is a videogame that has recently been shown to help cancer patients follow their drug regimen. Re-Mission is a “shooter” video game in which players destroy cancer cells using tools that evoke cancer therapies, such as chemotherapy. According to study published in the journal Pediatrics, patients who played for at least an hour a week were more likely to follow their drug regimen (PEDIATRICS Vol. 122 No. 2 August 2008, pp. e305-e317). Optimization of such interactive media may further improve the effectiveness of such interactive media in influencing behavior and attitudes.

Interactive media is often developed by iterative “user testing” in which a prototype product is refined based on behavioral and/or attitudinal measurements taken on people as they interact with that prototype. If product development is construed from the perspective of engineering control systems, this process involves optimization of a behavioral and/or attitudinal criterion. For example, a product may be designed by iterative alteration of a prototype product until it meets some observable behavioral criterion, such as users reporting that it is “easy to use” or “fun and enjoyable”, or such as developers observing that users spontaneously interact with the product for a specific duration or frequency, such as website visits. However, observable behavior or self report attitudinal indicators can be imperfect metrics for product optimization due to the “noise” inherent in measuring relevant behaviors, such as the imperfect correspondence between user experiences and the words available to express those experiences, user difficulties in recognizing or comprehending the importance of certain experiences, the “reactive measurement” effects of answering questions on the intrinsic experience of interacting with the prototype, imperfect correspondence between user experiences and observable nonverbal behavioral indicators of those experiences, etc.

Thus, it would be beneficial to provide interactive media, and methods of creating or operating interactive media that are optimized, particularly in their ability to evoke a desired behavior or attitude.

We propose herein an alternative strategy for interactive product optimization that is based on a use of one brain activity to help modify and optimize the design and/or use of the interactive media. In particular, described herein are methods of optimizing interactive media by modifying one or more optimizable elements of the interactive media based on comparison of the brain activity of a subject using the interactive media with criterion brain activity.

The use of brain activity may include the measurement of brain states and/or neural activity, including patterns of brain regional activity measured by functional methods such as functional Magnetic Resonance Imaging (fMRI) or Positron Emission Tomography (PET). Other metrics for determining brain activity may also be used, including electroencephalographic (EEG) recordings, and the like.

One major advantage of the approach described herein is the ability of product developers to measure users' subjective experiences without the potentially reactive effects of direct questioning. This approach may also provide more consistent or accurate measurements of user experiences because those experiences can become evident to product developers even if they are not recognized as relevant, remembered, or articulated in words by product users. Finally, the approach described herein can also be used when behavioral measures would be infeasible, or when there is no readily observable behavioral marker of a desired user experience, but that experience can be defined by a measurable neural state.

### SUMMARY OF THE INVENTION

In general, the methods of optimizing an interactive media described herein are based on a comparison of brain response (brain activity) during presentation of the interactive media with a criterion pattern or criterion patterns of brain activity. Also described herein is interactive media created and/or optimized using these methods. This interactive media may be optimized using the methods described herein during the development stage of the interactive media, or the interactive media described herein may include one or more of the optimization methods described herein during operation of the interactive media.

A criterion pattern of brain activity typically refers to the regional brain activity that is measured from one or more subjects that exhibits a desired behavior or attitude in relation to a target activity. A criterion pattern of brain activity may also refer to the regional brain activity that is measured from a single criterion subject and is correlated over time with changes in a desired behavior or attitude in relation to a target activity. A subject exhibiting a desired behavior or attitude in relation to a target activity may be referred to as a criterion subject. A target activity may be a therapy or an activity associated with a therapy. For example, the target activity or therapy may be a medical treatment, such as treatment of a disease or disorder (e.g., cancer therapy), or a healthful activity (such as smoking cessation, weight control, or the like). The target activity may also be a non-health related behavior such as purchasing a product (including purchasing the interactive media itself). Thus, the criterion pattern of brain activity may be determined from one or more subjects that exhibits the desired behavior (e.g., compliance with a medical therapy, not smoking, eating healthfully, etc.) relative to a target activity or therapy (e.g., medical treatment, smoking cessation, weight control, etc.). The criterion pattern of brain activity may also be determined from one or more subjects that exhibit the desired behavior (e.g., compliance with a medical therapy, not smoking, eating healthfully, etc.) relative to a target activity or therapy (e.g., medical treatment, smoking cessation, weight control, etc.) during certain periods, but not others.

The criterion brain activity may be a pattern of brain activity that is identified from such criterion subjects when the subjects are provided with a stimulus. Appropriate stimulus may include interacting with the interactive media. For example, a criterion pattern of brain behavior may be thought of as a typical response to the interactive media from a subject exhibiting the desired behavior. Any appropriate criterion pattern of brain activity may be used, including those that based on an expected pattern (or theoretical model) of brain activity, rather than those determined experimentally or recorded from criterion subjects. In some variations, the criterion pattern of brain activity is a pattern of brain activity that is identified from one or more criterion subjects when the subjects are not interacting with the interactive media to be optimized, but are interacting with some other stimulus (or no stimulus).

A criterion pattern of brain activity may be taken from a single subject or a population of subjects. In some variations, a set of criterion brain activity patterns may be generated, and applied to subjects having similar characteristics (e.g., age, gender, etc.) from the set of criterion brain activity patterns.

As used herein, “optimizing” an interactive media typically refers to the process of identifying the most effective presentation of one or more elements of the interactive media to evoke the desired behavior or attitude in a person after using the interactive media. Without being bound by a particular theory of action, the optimized interactive media may help train or motivate a person using the interactive media to achieve a desired behavior or attitude in relation to a target activity or therapy.

It may be particularly beneficial to have the interactive media relate in some way to the target activity or therapy. If the interactive media is a computer program such as a video game, the computer program may include features or elements that evoke the target activity. For example, if the target activity is a chemotherapy regime, the video game may include elements relating to chemotherapy, and the desired behavior or attitude exhibited by the criterion subjects is compliance with the chemotherapy regime. The behavior or desired attitude may also be referenced in the interactive media, either overtly or indirectly, e.g., by making administration of chemotherapy part of the game play.

In general, any appropriate interactive media may be used. For example, the interactive media may be software (e.g., computer programs, computer games, etc.), toys (including toys having electronic, software, hardware, firmware, etc.), websites or webpages, or the like.

An optimizable element may be any component of the interactive media that may be modified, including the look, sound (e.g., “feel”), gameplay, backstory, behavior of characters, setting, or the like. The optimizable element may be related to the desired/target behavior or attitude that the interactive media is being optimized towards. For example, the optimizable element may be ‘associated’ either directly or indirectly with the target behavior or attitude. In one variation, the target behavior or attitude is the compliance or motivation to comply with a therapeutic treatment such as chemotherapy, and the interactive media references the therapy. As an example, one of the optimizable elements may be a character representing a cancer cell, and the methods described herein may be used to optimized the look (e.g., shape, size, color, overall feel, etc.) and/or activity (movement, etc.) of this character. In other variations, the target behavior or attitude is the purchase or utilization of a product.

In one variation of the method of optimizing an interactive media based on brain response, the method includes the steps of: presenting a subject with a plurality of variations of the interactive media (each variation including an optimizable element in a different configuration), determining a pattern of brain activity from the subject during the presentation of each variation, comparing the pattern of brain activity during the presentation of each variation with a criterion pattern of brain activity, and optimizing the interactive media based on the comparisons between the criterion pattern of brain activity and the patterns of brain activity determined during the presentation of each variation.

The methods described herein may also include the step of defining the criterion pattern of brain activity. For example, the method may include the step of presenting a criterion subject with a stimulus and determining a criterion pattern of brain activity from the criterion subject, wherein the criterion subject exhibits a desired behavior or attitude with respect to a target activity or therapy. The stimulus may be the interactive media (e.g., a video game, website, or other interactive media). The interactive media may reference the target activity or therapy. As described above, the target activity may be an activity such as a medical therapy (e.g., cancer therapy), or an activity related to purchasing a product. In some variations the criterion pattern of brain activity is derived from more than one criterion subject. For example, the criterion pattern of brain activity may be derived from a population of subjects.

The stimulus used to evoke a criterion pattern of brain activity may be related to the interactive media (as mentioned, it may be a variation of the interactive media), or the stimulus may be related to the target activity or behavior. For example, the stimulus may be a real-life situation that is used to evoke the criterion pattern. If the target activity is smoking cessation, for example, the stimulus may be presentation of a cigarette to a subject that has successfully stopped smoking.

A criterion pattern of brain activity may also be determined indirectly. For example, a criterion pattern of brain activity may be generated based on published reports. A criterion pattern of brain activity may be determined by estimating activity for target brain regions believed to be related to certain behaviors or attitudes.

As mentioned, the interactive media may be any appropriate interactive media, including digital media such as video games. Thus, the step of presenting the subject with the interactive media may include presenting the subject with a video game and allowing the subject to play a video game. The subject may play the video game while brain activity is monitored. In some variations, the step of presenting the subject with the interactive media may include presenting the subject with a portion of the interactive media. For example, when the interactive media is a video game, the subject may be presented with a snapshot of the interactive media, or a portion of the interactive media (e.g., screen captures, partial play, etc.), rather than the complete interactive media. Alternatively the subject may be allowed to play the video game for a certain period of time, or for a certain portion of the game. In particular, the subject is presented with the portion of the interactive media including the optimizable element(s).

Thus, the method may include presenting the subject with variations of the interactive media that include an optimizable element in different configurations. For example, when the interactive media is a video game, the optimizable element may be a character in the video game (e.g., an opponent character), and the subject may be presented with variations of the character in a setting from the game, or as part of a partial or complete portion of game play. The different configurations may be differences in the character's appearance (size, look, etc.), differences in the character's actions (rate of movement, manner of movement, etc.), differences in the characters sound (sound effects, etc.), of the like. For example, if the optimizable element is the sound, music, etc., various sounds and/or music may be used at various portions of the presented game or other interactive media. Virtually any aspect of game play may be considered an optimizable element.

A pattern of brain activity may be determined in any appropriate manner, including imaging at least a portion of the subject's brain. When brain activity is imaged, the patterns of brain activity may be compared (or saved for later comparison) by comparing the image(s) to the criterion pattern of brain activity that is in a compatible modality (e.g., images or information extracted from the images). For example, the brain activity corresponding to the presentation of each variation of the optimizable element for the interactive media may be ‘subtracted’ from the criterion brain activity. This comparison may be quantified (e.g., by scoring the brain activity, and particularly the activity of certain regions). In some variations a ‘score’ based on the comparison of brain activity is generated by the comparison. The score may reflect which variation of the optimizable element results in brain activity closest to the criterion brain activity. The variation or option that results in a brain activity that most closely resembles the criterion may then be carried forward as the more ‘optimized’ variation. This variation may be accepted or further refined, by comparison with other variations. In some cases, variations of the optimizable element may be generated from the closest variation.

In general, brain activity patterns may be estimated, recorded, visualized, or the like. Appropriate modes of determining brain activity may include imaging, electrical measurements, etc., and the modality used to record from the test (optimizing) subjects may be the same as the mode used to measure or determine the criterion pattern. Imaging at least a portion of the subject's brain may comprise scanning the subject's brain by fMRI or PET. In some variations, brain activity is recorded at least partially by electrical recordings. For example, the step of comparing the pattern of brain activity may include recording an EEG from the patient and comparing the recorded EEG to an EEG representing the criterion pattern of brain activity.

Any appropriate number of optimization comparisons may be made by presenting one or more variations of the interactive media. In addition, any number of optimizable elements may be examined. Each variation of the interactive media presented to the subject may include one or more optimizable elements. For example, in some variations of the method, two or more optimizable elements are presented in different configurations in each variation of the interactive media presented. The different configurations of an optimizable element or elements may be compared all at the same time, or in combinations. Thus, it should be understood that multiple elements (or multiple features of a single element) may be presented and optimized concurrently. The resulting brain activity may be compared with the criterion brain activity. As will be apparent to those of skill in the art, statistical analysis of the resulting comparison with the criterion brain activity may be used to further optimize, or to guide the optimization. The resulting multivariate optimization may include different configurations of multiple elements and/or different features of the elements, and the effects in different brain regions and the intensity of activity in different brain regions, particularly in variations in which brain activity is functionally imaged.

The determination of which variations of interactive media to present (each variation of the interactive media having a different configuration of one or more optimizable element) may be guided by the results of comparisons of previously presented variations of the interactive media. For example, when the brain activity evoked by two different configurations of an optimizable element are compared to the criterion brain activity pattern, subsequent variations of the interactive media may be presented that include configurations of the optimizable element evoking a pattern of brain activity more like the criterion pattern. For example, if the optimizable element is the loudness of a tone or sound, the comparison of brain activity evoked by a soft and slightly louder element with the criterion brain activity may indicate if it would be best to present a softer or louder variation next.

The step of optimizing the configuration of the optimizable element may selecting a configuration that was presented to the subject that evokes a pattern of brain activity most closely resembling the criterion pattern of brain activity, or it may include estimating a configuration based on the configuration (or configurations) that evoke patterns of brain activity that are closest to the criterion pattern. Thus, the optimization method does not require that the optimized element have the same configuration as any of the specific tested configurations, but may be based on directional extrapolation from the observed results. For example, the optimizable element may be the number of “foes” in a video game. Two variations of the game may be presented; in the first variation, the configuration of foes includes two foes, and in the second variation, the configuration of foes includes four foes. A comparison of the brain activity evoked by these different variations with a criterion pattern of brain activity may indicate that the presentation of four foes results in a pattern of brain activity that best approximates the criterion pattern. Based on this comparison, the videogame may be optimized to have 6 foes, which is a configuration that was never specifically tested, but which was selected based on an inference drawn from the configurations that were tested, indicating that, in general, more foes is better.

In another variation of the methods described herein, the method of optimizing an interactive media based on brain response may include the steps of: presenting a subject with the interactive media including an optimizable element in a first configuration; comparing the pattern of brain activity during the presentation of the first configuration of the optimizable element with a criterion pattern of brain activity; presenting the subject with the interactive media including a second configuration of the optimizable element; comparing the pattern of brain activity during the presentation of the second configuration of the optimizable element with the criterion pattern of brain activity; and optimizing the interactive media based on the comparisons between the criterion pattern of brain activity and the pattern of brain activity during the presentation of the first configuration of the optimizable element and the second configuration of the optimizable element.

Also described herein are methods of optimizing an interactive media based on brain response. This methods may include the steps of: determining a criterion pattern of brain activity; presenting a subject with the interactive media including an optimizable element in a first configuration; assessing a brain activity pattern corresponding to the presentation of the first configuration; presenting the subject with the interactive media including a second configuration of the optimizable element; assessing a brain activity pattern corresponding to the presentation of the second configuration; determining if presentation of either the first configuration or the second configuration results in a brain activity pattern that is most similar to the criterion pattern of brain activity; and optimizing the interactive media to include the configuration of the optimizable element that results in a brain activity pattern that is most similar to the criterion pattern of brain activity. These methods may be similar to those already described, and may include any of the steps or features described above.

For example, the step of determining a criterion pattern of brain activity may include presenting a criterion subject with the interactive media and determining a criterion pattern of brain activity from the criterion subject, wherein the criterion subject exhibits a desired behavior or attitude with respect to a target activity or therapy, and further wherein the interactive media references the target activity or therapy.

Also described are methods of optimizing an interactive media based on brain response, the method comprising the steps of: presenting a criterion subject with the interactive media and determining a criterion pattern of brain activity from the criterion subject, wherein the criterion subject exhibits a desired behavior or attitude with respect to a target activity or therapy, further wherein the interactive media references the target activity or therapy; presenting a second subject with the interactive media including an optimizable element in a first configuration and assessing a brain activity pattern corresponding to the presentation of the first configuration; presenting the second subject with the interactive media including a second configuration of the optimizable element and assessing a brain activity pattern corresponding to the presentation of the second configuration; optimizing the interactive media to include the configuration of the optimizable element that results in a brain activity pattern that is most similar to the criterion pattern of brain activity.

In addition to the methods for optimizing interactive media described above, interactive media produced by one or more of the methods described above are also included herein. Thus, described herein are video games optimized using one or more of the methods described herein. For example, a video game optimized based on brain response(s) from a criterion subject demonstrating a desired behavior or attitude are described herein. Such a video game may be directed to helping improve a medical therapy (or compliance with a medical therapy) based on optimization by comparison with brain activity from a criterion subject that has a positive attitude or a high compliance with the medial therapy. The interactive media (e.g., video game) may include overt or symbolic aspects or elements of the medical therapy.

### BRIEF DESCRIPTION OF THE DRAWINGS

FIG. 1 is a flow diagram illustrating one variation of a process for optimizing interactive media.

FIG. 2 is a diagram showing one variation of a process for optimizing interactive media based on functional neural activity of users.

FIG. 3 illustrates schematically an example of a process applied to production of a videogame intended to alter cancer-prevention attitudes.

FIGS. 4A-4E illustrates one variation of a method for optimizing an interactive product based on fMRI (functional Magnetic Resonance Imaging) of brain activity.

FIGS. 5A-5E reproduce the example shown in FIGS. 4A-4E without the lines indicating the active regions.

### DETAILED DESCRIPTION OF THE INVENTION

Described herein are methods of optimizing or improving interactive media products based on brain response so that the interactive media may promote a user of the interactive media to behave or be motivated towards a target behavior or attitude. The interactive media may be a digital media such as a videogame or webpage. The target behavior or attitude may be any target behavior, including health-related behaviors or attitudes (e.g., compliance with a medical therapy, controlling weight, smoking cessation, etc.) and commercially relevant behaviors or attitudes, such as consumer purchasing choices. In many of the variations described herein, the subject of the target behavior or attitude is either overtly or symbolically related to the content of the interactive media.

In general, these methods for optimizing interactive media include the presenting a user (e.g., a “subject”) with different variations of the interactive media presenting different configuration of an optimizable element, and comparing the resulting brain activity pattern with a criterion pattern of brain activity. By comparing the evoked pattern of brain activity from each configuration to a criterion pattern, the optimizable element (and thus the interactive media) may be optimized by selecting a configuration that will evoke a pattern of brain activity that is closest to the criterion pattern of brain activity. The criterion pattern of brain activity may be chosen because it is correlated with the target behavior or attitude, as described in greater detail below.

The following description illustrates one example of interactive media, a videogame called “Re-Mission”, which is a “shooter” video game in which players destroy cancer cells using tools that evoke cancer therapies, such as chemotherapy. Re-Mission is intended for young people with cancer. The examples provided below illustrate various ways that an interactive medium such as the Re-Mission video game may be optimized to promote a user to be motivated to comply with a therapeutic regime such as chemotherapy.

The methods for optimizing an interactive media described herein are not limited to Re-Mission, however, and may be used to optimize any appropriate interactive media. Optimization may be performed for an audience, for an individual, or for a targeted audience. For example, the interactive media may be optimized for an individual by determining the criterion pattern of brain activity from that individual. In other variations of the method of optimization, an interactive media may be optimized to a specific target audience, such as 14-18 year old males, by selecting a criterion pattern of brain activity from a criterion subject (or subjects) within this target audience. Thus, the criterion pattern of brain activity may be matched to the target audience.

As mentioned, the optimization of the interactive media may promote a user of the interactive media to behave or be motivated towards a target behavior or attitude. An interactive media may be optimized to promote social behaviors that are not directly related to health (e.g., promoting voting, good driving, politeness or tolerance, school attendance, etc.). Similarly, the interactive media may be optimized to promote the consumption of a product (including purchase of a product that is referenced in the interactive media, or purchase of the interactive media product itself).

Without being bound to a particular theory of operation, the methods described herein may help motivate a subject towards a target behavior or attitude by allowing the subject experience a brain pattern during operation of the interactive media (e.g., during game play) that is similar or identical to the brain pattern experienced in a similar context by a criterion subject that demonstrates the target behavior or attitude. Thereafter the test subject may be more likely to act like the criterion subject in a situation related to the theme of the interactive media. In essence, the subject's experience of a brain pattern that is experienced by someone who exhibits a target behavior or attitude (even only while performing a symbolically related task) may lead or allow the subject to later experience a similar behavior or attitude. The interactive media may be used to recapitulate a criterion brain state and thereby may lead to a recapitulation of state-associated behavior. There is evidence that recapitulating neural activity using chemical or electrical means can reliably recapitulate some simple behaviors (e.g., electrically stimulating specific brain regions relatively reliably evokes certain visual experiences, muscular movements, word production, etc.). However, the use of symbolic stimuli such as interactive media to evoke a brain state may be more complex, because many of the structures activated by the symbolic stimulus (e.g., visual cortex) may not be strictly required for the desired behavior. Also, many psychological drivers of complex behaviors such as chemotherapy adherence (e.g., attitudes, memories, beliefs) may not yet be directly observable using conventional fMRI or any other brain imaging technology. Thus, although it may be uncertain exactly which brain states need to be achieved to create a long-term propensity to behave in a certain way, the methods described herein may still provide optimization based on brain activity that is correlated with subsequent attitudes and behavior. These methods may be further enhanced by focally evoking brain activity patterns related to the motivation, but relatively unrelated to the peripheral activity (i.e., independent of other brain activity involved in manipulating the interactive media such as playing a videogame). Such focally evoke brain activity patterns may be used as part of the criterion brain pattern, for example.

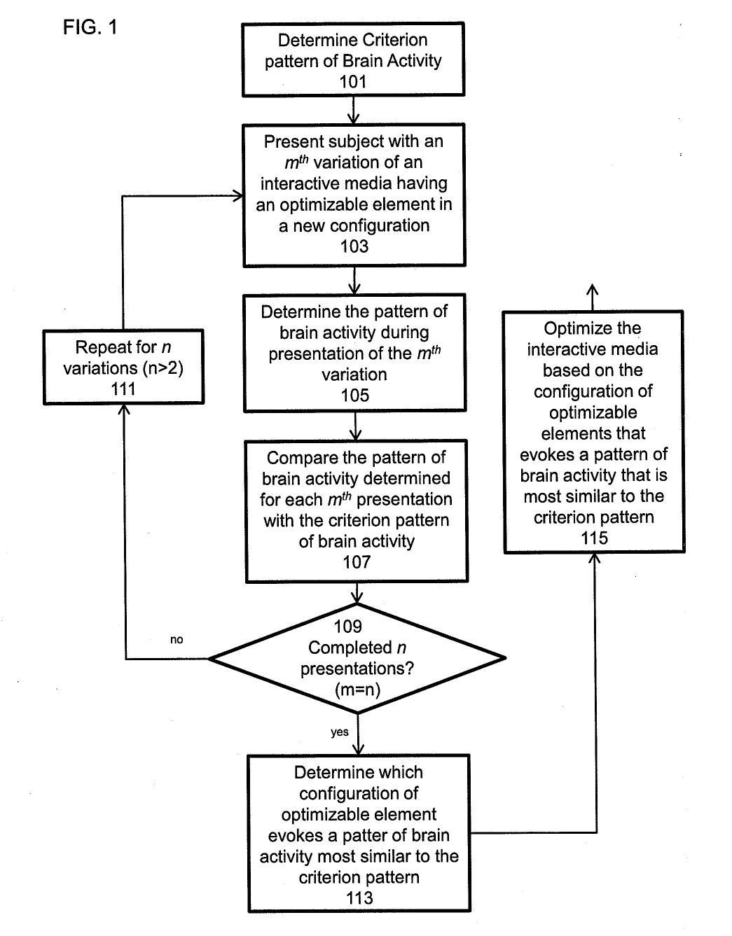

FIG. 1 provides an overview of one variation of a method for optimizing an interactive media. In the first (optional) step shown in FIG. 1, a criterion pattern of brain activity is defined 101. As mentioned above, a criterion pattern of brain activity may be defined in any appropriate manner. For example, a criterion pattern of brain activity may be recorded from a criterion subject, where the subject exhibits the target behavior or attitude. A criterion pattern of brain activity may be recorded from the criterion subject after providing a stimulus. For example, the stimulus may be exposure to the interactive media. Other stimuli may include exposure to a subject relevant to the target behavior. For example, if the target behavior or attitude is weight control or restraint, the stimulus may be exposure to food.

In the exemplary method described in FIG. 1, the next series of steps include presentation of a plurality of variations of the interactive media 103 and determining the pattern of brain activity during each presentation 105. Each variation of the interactive media may include one or more optimizable element that is in a configuration that is different between the different variations. The presentation step may include displaying the interactive media and may also include allowing the user to interact with the interactive media. For example, if the interactive media is a webpage, the user may be shown the webpage (or a portion of the webpage) and allowed to interact by clicking one or more links. If the interactive media is a video game, the user may be shown all or a portion of the video game; the user may also be allowed to play the video game or a portion thereof.

The pattern of brain activity for a subject (“user”) during the presentation of the interactive media variations may be determined in any appropriate manner. For example, the pattern may be determined by imaging all or a portion of the users brain, and particularly functional imaging (e.g., fMRI, PET, etc.). In some variations brain activity may be determined from one or more electrodes (e.g., EEG). Any combination of these methods of determining brain activity may be used. Brain activity may be recorded, or it may be analyzed immediately. For example, brain activity may be compared to the criterion brain activity pattern without recording it. In some variations the brain activity is analyzed and the results of the analysis (e.g., measurements of amplitude, regions of change, or the like) stored or used for the later comparison.

Although FIG. 1 illustrates one version of the method in which the pattern of brain activity evoked by a variation of the interactive media is compared to the criterion pattern of brain activity before the presentation of the next pattern of brain activity 107, this step may instead occur after all or a subset of the interactive media variations have been presented. For example, the patterns of brain activity evoked during the presentation of the interactive media variations may be stored (or analyzed to determine metric characteristics that are stored), and later compared to the criterion pattern of brain activity.

In the method shown in FIG. 1, the steps of presenting variations of the interactive media 103 and determining the evoked pattern of brain activity 105 are repeated 111 until all of the variations of the interactive media (“n variations”) have been shown 109.

In some versions of the method, the number of variations of the interactive media (n), that are included is variable. For example, a new variation of the interactive media may be created (an “m^{th}” version) and tested based on the results of the comparison of previous variations of the interactive media with the criterion pattern. Thus, different configurations of the optimizable element may be tested based on the response evoked by other configurations. Alternatively, a fixed number (n) of variations of the interactive media may be tested. In some variations, a fixed array of variations may be prepared, however the response of earlier-presented variations may limit or eliminate the need to present later variations; in particular, if a trend of the effect of changing the configuration of the optimizable element becomes apparent.

Returning to FIG. 1, once the variations have been presented and the evoked patterns of brain activity compared to the criterion pattern of brain activity, the configuration that evokes a pattern most similar to the criterion pattern may be determined 113. Thereafter, the interactive media may be optimized based at least in part on the variation of the interactive media that evokes a pattern most similar to the criterion pattern. In some versions of the method, the configuration of the optimizable element that evokes the brain activity pattern most similar to the criterion brain activity pattern is chosen for the interactive media. In some versions, the optimizable criterion is determined based on a pattern of similarity to the criterion brain activity pattern for the array of different configurations of the optimizable element presented.

It should be noted that an optimizable element of an interactive brain activity may be any element of the interactive media that may be modified; elements may include multiple features which may themselves be elements. For example, the optimizable element may be a feature in the video game such as the background; the background may include multiple elements such as color, texture, lighting, depth, etc. Variations of any of these may form different configurations of the element that may be tested and compared.

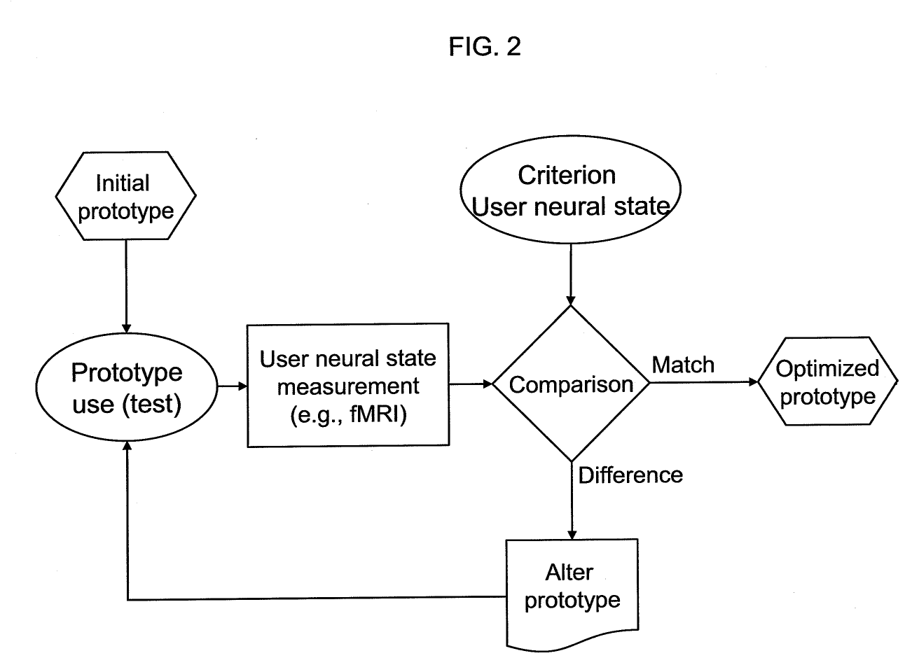

FIG. 2 illustrates an overview of one version of a process for optimizing an interactive media based on the functional neural activity of the users. In this example, an initial prototype is iteratively modified by comparisons between the neural states (pattern of brain activity) and a criterion user neural state (the criterion pattern of brain activity). If there is a ‘match’ (within a predetermined tolerance range, for example), the most recent variation of the prototype interactive media is deemed the ‘optimized’ variation. If there is no match, the prototype may be again altered, and presented to the user so that the evoked neural state can be again compared to the criterion neural state. The tolerance set for determining a “match” may be adjusted or adjustable. The lower the tolerance, the more similar the patterns are required to be. In any of the comparison steps described herein, the comparison may be weighted. For example, a match may be required in certain brain regions or certain characteristic portions of the brain activity pattern (e.g., theta waves), while other portions or regions of the brain activity response may be ignored completely or weighted much lower. The determination of weighting may be based on experimental or theoretical principles. For example, regions of the brain known to be involved in the interactive response (e.g., motor cortex) and less important for motivation may be weighted lower than other brain regions. An examination across populations of criterion subjects may help provide weighting to the comparison, in addition to determining a criterion pattern of brain activity. For example, if comparing across a plurality of criterion subjects, regions having a higher variation between subjects may be weighted much lower than regions that are highly coincident.

As described above, in the optimization methods described herein the product to be optimized under the present claim is an interactive product (e.g., a toy, computer program, website, videogame, etc., in which the user influences the content, form, or activity of the product in some way). The method of optimization typically includes intentional iterative refinement of the technology based on the evoked pattern of brain activity. FIGS. 1 and 2 illustrate the feedback relationship between measured neural activity profiles and further product development.

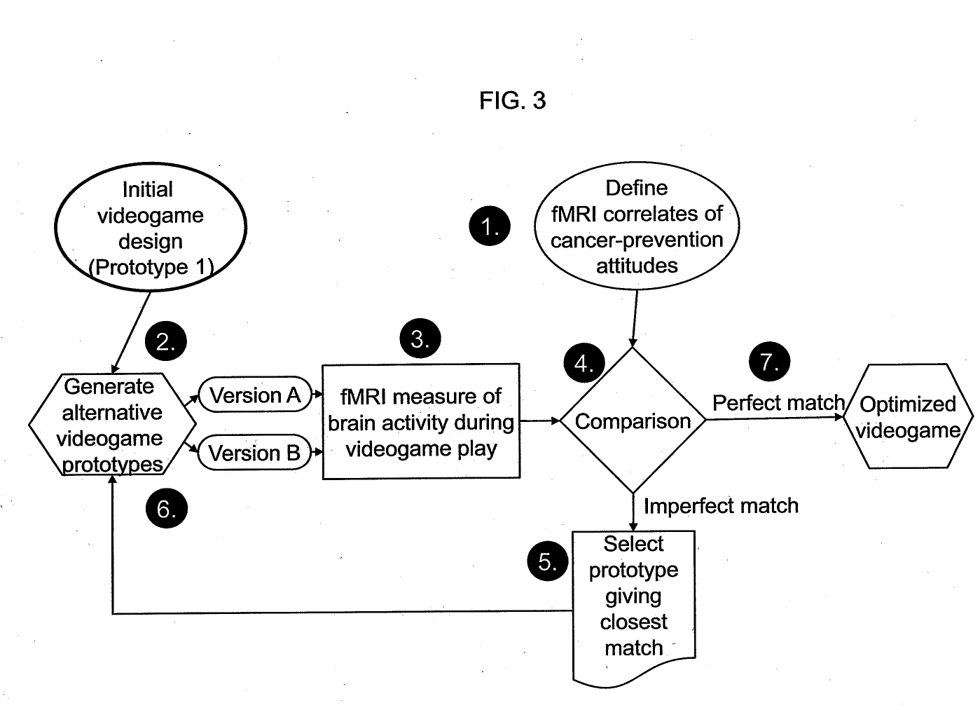

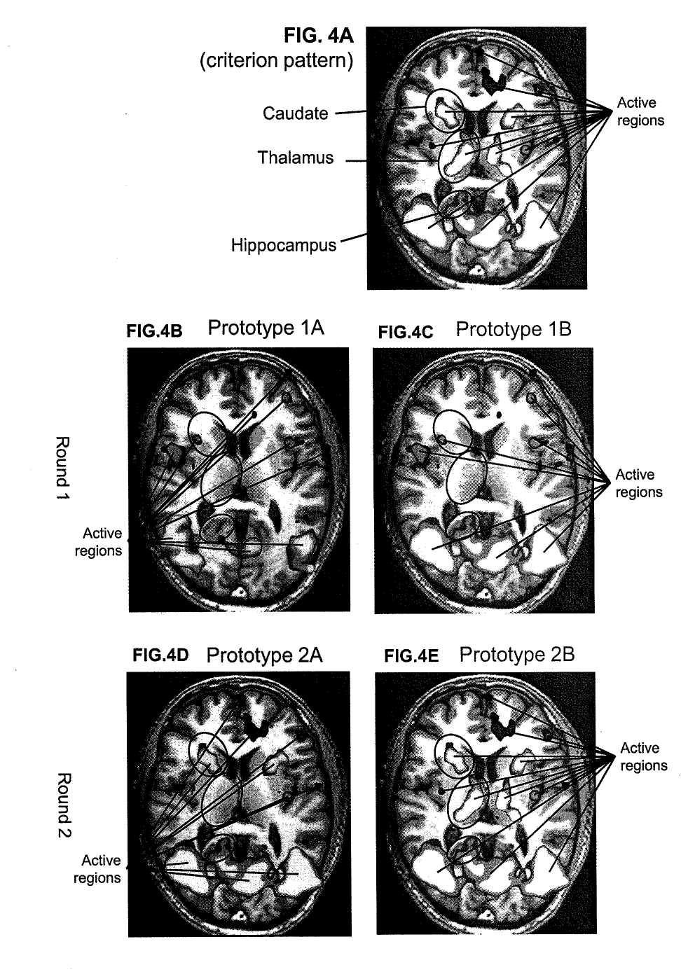

For example, FIG. 3 provides an example of the optimization process applied to the development of the Re-Mission videogame that is intended to alter young people's attitudes regarding cancer-prevention behavior. In general, accurate measurement of attitudes by behavioral measures or self-report is complicated by absence of precise language metrics for quantifying attitudes, social desirability biases in people's self reports of their attitudes, and the difficulty in comprehensively monitoring cancer-related nonverbal behaviors that might serve as indicators of attitudes. In Step 1 of the exemplary method shown in FIG. 3, the product developer carries out an intensive pilot study to identify brain activity correlates of cancer prevention-related attitudes. Referring now to FIG. 4A, a regional brain activity pattern identified by fMRI imaging is illustrated. This pattern of brain activity is believed to be a good correlate of cancer-related attitudes.

The profile of brain activity shown in FIG. 4A thus serves as the criterion of brain activity to be achieved (optimized) in the iterative development of the video game. The data shown represents profiles of functional neural activity measured by fMRI in people with a strong positive attitude towards cancer-prevention behavior, while they are playing part of the Re-Mission videogame. Thus, this profile may serve as the “optimal” criterion for the development of a different videogame tool (e.g., a new version of Re-Mission) to increase positive attitudes towards cancer treatment.

In this example, the criterion imaging (e.g., determining a criterion pattern of brain activity) is performed by having criterion subjects play the Re-Mission videogame, and then measuring attitudes toward cancer and chemotherapy afterward (both immediately after game play and 1 month later). A positive attitude toward chemotherapy is the “target behavior”, in part because studies have linked positive attitudes toward medications to increased adherence to prescribed medication regimens.

In the criterion-establishing fMRI study, patterns of brain activity were analyzed as subjects played Re-Mission, and data analysis was used to determine which brain regions show activation that is correlated with subsequently measured attitudes toward chemotherapy. The “target behavior” is attitude toward chemotherapy, and the “brain activity criterion” is the profile of brain regions whose activation during gameplay is correlated with the target behavior.

The intensity maps (indicated in the black and white reproductions shown in FIG. 4A-4E by lines pointing to the regions of activity) mark regions of the brain that showed positive correlation between gameplay-induced activation and positive attitudes toward chemotherapy. In FIGS. 4A-4E, the activity is indicated by outlined regions overlaying the brain section. Such scans are normally visualized in color, in which pseudocoloring may indicate the intensity of the brain activity in various regions. In FIG. 4A specifically, regions of the caudate, putamen, thalamus, and hippocampus (circled) showed activation during game-play, and the relative magnitude of that activation for a given participant was correlated with the intensity of that participant's positive attitude toward chemotherapy. As such, that profile of “caudate/putamen/thalamus/hippocampus” activation has been adopted as the criterion brain activity. Note that the brain activity criterion could have been chosen using a different method that did not involve playing a videogame. In this case, both the “criterion-defining stimulus” and the thing to be optimized on the basis of that criterion are both videogames.

Following the additional method steps (and particularly those outlined in FIG. 3), to optimize the Re-Mission game to maximally enhance adherence to chemotherapy, the game may be optimized by iteratively determining which configuration of elements, from a menu of possible configurations, most strongly activates the caudate/putamen/thalamus/hippocampus region in the manner most similar to the criterion pattern identified as just described.

Participants in the initial criteria-defining study were undergraduate students recruited through fliers posted at a university (i.e., essentially at random), assuming that they would be broadly representative of the young adult demographic to which the intervention products are targeted. Alternatively, criterion subjects may be recruited from the target audience (e.g., adolescent and young adult cancer patients).

Returning now to the exemplary method shown in FIG., 3, in step 2, alternative variants of the game are developed (e.g., altering one element or aspect of the videogame, such as the appearance of characters), and in Step 3, functional neural activity patterns are assessed by fMRI imaging while young people in the target audience demographic play one of two alternative versions of the videogame. FIG. 4B and 4C illustrate exemplary fMRI regional brain activity patterns generated as users play two different Round 1 prototypes of the video game; Prototypes 1A and 1B. In this example, the prototypes differ in their depiction of cancer cells (e.g., “funny” vs. “mean”). Step 4 of the method shown in FIG. 3 involves comparison of the regional functional neural activity profile generated by each prototype version with the criterion neural activity profile. If none of the existing prototype versions generates exactly the same regional activity profile as the criterion, Step 5 selects that variant which most closely approximates the criterion. In this example, FIG. 4C more closely approximates the criterion brain activity profile (activation of the caudate, thalamus, and hippocampus). Prototype 1A is excluded and Prototype 1B is advanced to the next stage of development. In Step 6 of the method shown in FIG. 3, the optimal variant emerging from Round 1 analysis is subject to further alterations in Round 2 of development. For example, two different variants of Prototype 1B might be generated in which cancer cells are “mean” in each (i.e., both variants are optimized on Round 1 criteria), but in Prototype 2A they move slowly and clumsily whereas in Prototype 2B they move quickly and aggressively. FIGS. 4D and 4E illustrate fMRI results from Round 2 testing indicating that Prototype 2B best approximates the criterion pattern of functional neural activity (that shown in FIG. 4A). This iterative continues until either: (1) the criterion brain state is perfectly achieved by a prototype, which is then taken as the optimized finished product in Step 7, or (2) all available dimensions of product variation have been parametrically varied, and the prototype videogame most closely approximating the criterion brain state is selected for transformation into a finished product in Step 7. In this example, fMRI measurements of a target brain state are used as a “functional neural biomarker”—an objectively measured neurobiological proxy for an ideal user state that is more difficult to measure using other available means (i.e., cancer-related attitude).

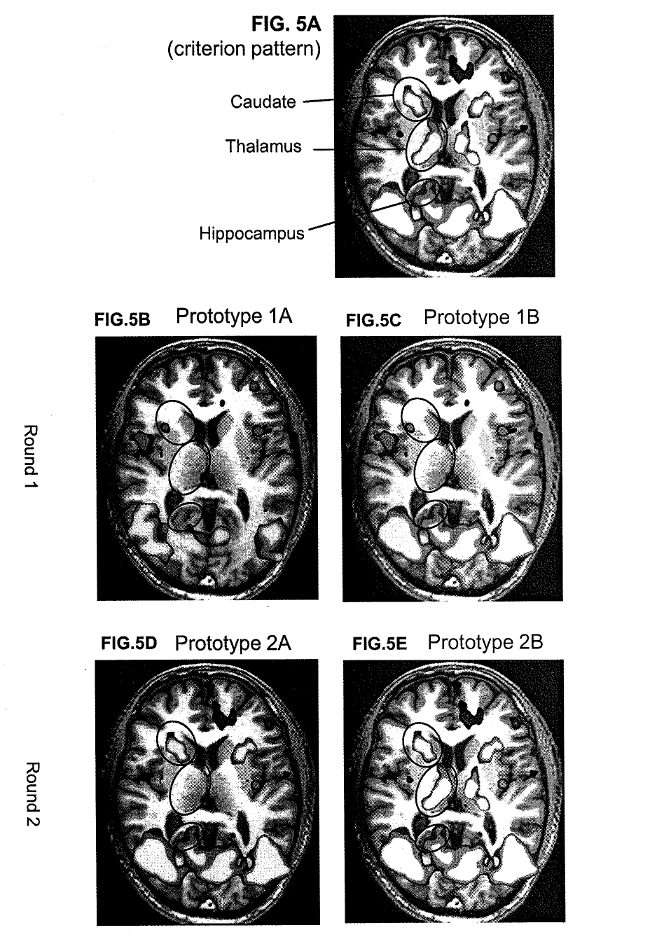

The scans shown in FIGS. 4B-4E illustrate exemplary, rather than experimental, patterns of brain activity. FIGS. 5A-5E show the same figures of FIGS. 4A-4E with the lines (pointing to regions of activity) removed. The different patterns of brain activity can be readily identified in FIGS. 5A-5E using the lines shown in FIG. 4A-4E as a guide.

While the methods for optimizing an interactive media, as well as interactive media optimized using these methods, have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

We claim as our invention:

1. A method of optimizing an interactive media based on brain response, the method comprising the steps of:
presenting a subject with a plurality of variations of the interactive media, each variation including an optimizable element in a different configuration;
determining a pattern of brain activity from the subject during the presentation of each variation;
comparing the pattern of brain activity during the presentation of each variation with a criterion pattern of brain activity; and
optimizing the interactive media based on the comparisons between the criterion pattern of brain activity and the patterns of brain activity determined during the presentation of each variation.

2. The method of claim 1, further comprising defining the criterion pattern of brain activity.

3. The method of claim 1, further comprising the step of presenting a criterion subject with the interactive media and determining a criterion pattern of brain activity from the criterion subject, wherein the criterion subject exhibits a desired behavior or attitude with respect to a target activity or therapy.

4. The method of claim 1, wherein the interactive media references a target activity or therapy that is related to the criterion pattern of brain activity.

5. The method of claim 1, wherein the interactive media is a video game.

6. The method of claim 1, wherein the step of presenting the subject with a plurality of variations of interactive media including an optimizable element comprises presenting a plurality of variations of a video game wherein each variation includes an different variation of one or more optimizable element.

7. The method of claim 1, wherein the step of determining the pattern of brain activity from the subject comprises imaging at least a portion of the subject's brain.

8. The method of claim 7, wherein the step of imaging at least a portion of the subject's brain comprises scanning the subject's brain by fMRI or PET.

9. The method of claim 1, wherein the step of determining the pattern of brain activity from the subject comprises recording an EEG.

10. The method of claim 1, wherein the step of optimizing the interactive media comprises selecting whichever configuration of the optimizable element from the plurality of variations of the interactive media evokes a pattern of brain activity that is most similar to the criterion pattern of brain activity.

11. The method of claim 1, wherein the step of optimizing the interactive media comprises generating a configuration of the optimizable element based on whichever configuration of the optimizable element evokes a pattern of brain activity that is most similar to the criterion pattern of brain activity.

12. A method of optimizing an interactive media based on brain response, the method comprising the steps of:
presenting a subject with the interactive media including an optimizable element in a first configuration;
comparing the pattern of brain activity during the presentation of the first configuration of the optimizable element with a criterion pattern of brain activity;
presenting the subject with the interactive media including a second configuration of the optimizable element;
comparing the pattern of brain activity during the presentation of the second configuration of the optimizable element with the criterion pattern of brain activity; and
optimizing the interactive media based on the comparisons between the criterion pattern of brain activity and the pattern of brain activity during the presentation of the first configuration of the optimizable element and the second configuration of the optimizable element.

13. A method of optimizing an interactive media based on brain response, the method comprising the steps of:
determining a criterion pattern of brain activity;
determining a pattern of brain activity from a subject during the presentation of each of a plurality of variations of the interactive media, wherein each variation including an optimizable element in a different configuration;
comparing the pattern of brain activity during the presentation of each variation with a criterion pattern of brain activity;
determining which variation of the interactive media presented that results in a brain activity pattern that is most similar to the criterion pattern of brain activity; and
optimizing the interactive media based on the comparisons between the criterion pattern of brain activity and the patterns of brain activity determined during the presentation of each variation.

14. The method of claim 13, wherein the step of determining a criterion pattern of brain activity comprises presenting a criterion subject with a stimulus so that the criterion subject exhibits a desired behavior or attitude with respect to a target activity, and recording the evoked pattern of brain activity from the criterion subject.

15. The method of claim 14, wherein the stimulus comprises a presentation of the interactive media.

16. The method of claim 13, wherein the interactive media comprises a video game.

17. The method of claim 14 wherein both the stimulus and the interactive media reference a common target activity.

18. The method of claim 17, wherein the common target activity comprises a medical treatment.

19. The method of claim 17, wherein the common target activity comprises purchasing a product.

20. The method of claim 13, wherein the step of determining a pattern of brain activity comprises scanning the subject's brain by fMRI or PET, or recording an EEG.

21. A method of optimizing an interactive media based on brain response, the method comprising the steps of:
presenting a criterion subject with a stimulus and determining a criterion pattern of brain activity from the criterion subject, wherein the criterion subject exhibits a desired behavior or attitude with respect to a target activity or therapy, further wherein the interactive media references the target activity or therapy;
presenting a second subject with the interactive media including an optimizable element in a first configuration and assessing a brain activity pattern corresponding to the presentation of the first configuration;
presenting the second subject with the interactive media including a second configuration of the optimizable element and assessing a brain activity pattern corresponding to the presentation of the second configuration;
optimizing the interactive media to include the configuration of the optimizable element that is related to the configuration of the optimizable element that results in a brain activity pattern that is most similar to the criterion pattern of brain activity.

22. The method of claim 21, wherein the steps of presenting the criterion or second subject with the interactive media comprise allowing the subject to play a video game.

23. The method of claim 21, wherein the steps of assessing the brain activity pattern comprises taking one or more of: fMRI scan, PET scan or EEG recording.
